(19) United States
(12) Patent Application Publication
(10) Pub. No.: US 2025/0197366 A1
(43) Pub. Date: Jun. 19, 2025

(54) TOFA ANALOGS AND THEIR USE FOR REDUCING OR PREVENTING SEBUM

(71) Applicant: BEIERSDORF AG, Hamburg (DE)

(72) Inventors: Judith SEIDEL, Berlin (DE); Joern Hendrik REUTER, Henstedt Ulzburg (DE); Ahmed KAMAL, Hamburg (DE); Annika SIMMERING, Hamburg (DE); Heiko MIELKE, Neu Wulmstorf (DE)

(21) Appl. No.: 18/835,209

(22) PCT Filed: Jan. 16, 2023
(86) PCT No.: PCT/EP2023/050869

§ 371 Date: Aug. 1, 2024

(30) Foreign Application Priority Data
Feb. 8, 2022 (DE) 10 2022 201 277.1

(51) Int. Cl. C07D 307/68; A61K 8/49; A61K 31/341; A61P 17/08; A61Q 5/00; A61Q 19/00

(52) U.S. Cl. CPC C07D 307/68; A61K 8/4973; A61K 31/341; A61P 17/08; A61Q 5/008; A61Q 19/008

(57) ABSTRACT

Compounds of general formula (1)

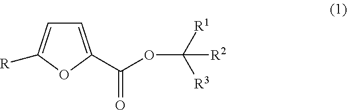

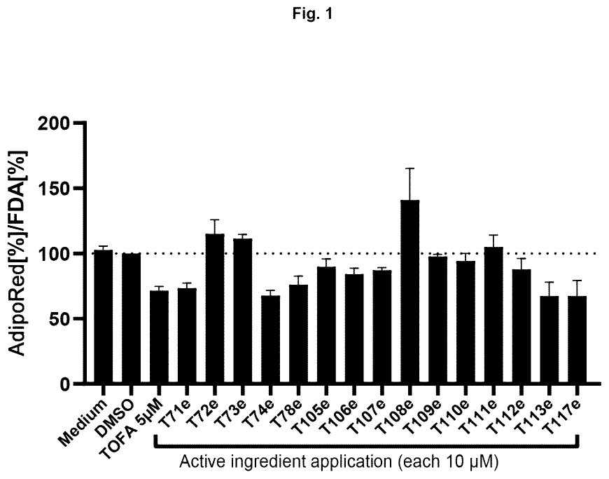

The present invention relates to novel substances and uses thereof as active ingredients and preparations containing such active ingredients which are effective against greasy and/or impure skin, and mild forms of acne and/or in the case of greasy hair.

Seborrhea is a predisposition to increased function of the sebaceous glands. Both scalp and facial skin appear greasy. The composition of seborrheic sebum is altered compared to normal sebum. Three stages of development of seborrhea are distinguished:
1. Simple seborrhea: mild cases, greasy after 8 days.
2. Oily seborrhea: already greasy after 2-3 days.
3. Irreversible form: no longer reversible. Seborrhea where the hair looks like it has been bathed in grease after just one day.

The excessive secretion of the sebaceous glands can, inter alia, be triggered by androgenetic disorders (male sex hormone disorder) and has a detrimental esthetic effect on the overall appearance of the hair. This disorder can also be the cause of hair loss that occurs. The precursor in each case is the seborrhocic condition of the scalp. Vegetative disorders and improper care can worsen the appearance of the skin and also the condition of the hair. Even with seborrhea, the hair itself can be dry due to disturbances in keratin formation. Dry, weakened hair is often caused by external stress such as sun or chemical treatments. Excessively hot blow-drying or not properly caring for weakened hair can lead to damage.

The causes of greasy hair lie in the human body and are hormonal. Each hair has its own sebaceous gland, which produces grease (also called sebum). Sebum production is hormonally controlled, and over- or underproduction can occur, depending on the hormone sensitivity of the sebaceous gland. The sebum itself has the function of keeping the scalp supple. It passes from the sebaceous gland to the scalp and only later the hair root. There it is normally absorbed by the hair shaft and remains invisible. When sebum is overproduced, the hair shaft is no longer able to absorb it. It becomes visible as a greasy film on the hair. The result is stringy hair that is shiny with grease.

Due to the fact that sebaceous gland production is dependent on the hormone balance, the problem of greasy hair cannot be solved fundamentally since the sebaceous glands continually produce grease. Consistent care and high-quality skin care products are still the best way to combat greasy hair.

Unfortunately, greasy hair has very troublesome effects. The hair becomes stringy again just a short time after washing and the hairstyle does not hold.

Contrary to popular belief, it is only a rumor that the hair becomes greasy more quickly as a result of excessively frequent washing. Mild shampoos for greasy hair ensure that excess grease is removed. Hair and scalp are supplied with sufficient moisture and balance the overproduction of the sebaceous glands.

Greasy hair and dandruff are among the most common hair problems. These abnormalities can be attributed to a disorder in the activity of the sebaceous glands. If the sebaceous glands are hyperactive, the term used is seborrhea. Two forms can be distinguished here: the oily form (seborrhea oleosa) and the dry form (seborrhea sicca).

### Seborrhea Oleosa:

This involves hyperactivity of the sebaceous glands, where the sebaceous glands produce too much, and too oily, sebum. The skin therefore has a greasy sheen, and the hair is greasy and stringy again right down to the tips just 2 to 3 days after washing.

### Seborrhea Sicca:

This can likewise be attributed to hyperactivity of the sebaceous glands, but the skin sebum is drier and has a firmer consistency. With the small flakes of the epidermis, it forms large, easily friable sebum flakes. The scalp has a waxy sheen, the hair becomes greasy only at the roots, and the lengths and particularly the tips are dry and even brittle.

The treatment of seborrhea includes, first of all, regular and thorough washing of the head with special shampoos, which can be carried out as often as seems necessary. The washing should be combined with massage in the connective tissue because this empties the sebaceous glands more, which delays the regreasing.

In the case of impure skin and mild forms of acne, in addition to increased sebum production, other influences such as secondary bacterial infections are also of etiological importance. One of the most important microorganisms associated with impure skin is Propionibacterium acnes.

Impure skin and/or comedones adversely affect the well-being of those affected, even in mild cases. Since practically every adolescent is affected by impure skin to some degree, for many people there is a need to remedy this condition. Since a major factor here is the excessive production of sebum, in many cases it is possible to improve the condition of the skin just through the use of sebum-reducing active ingredients.

It was thus the object of the present invention to find preparations that are effective against greasy and/or impure skin, mild forms of acne and against greasy hair.

The prior art already discloses some attempts to solve this problem, but they do not disclose all the advantages of the development presented herein.

WO 2011/005660 discloses analogs of 5-(tetradecyloxy)-2-furancarboxylic acid (TOFA) and their antiseborrheic effect.

Document WO 2018/022797 discloses analogs of 5-(tetradecyloxy)-2-furancarboxylic acid (TOFA) and their effect against acne vulgaris.

Document WO 2019/115405 discloses analogs of pyrrole derivatives and their antiseborrheic effect.

However, it has surprisingly been found, and therein lies the achievement of the object, that novel compounds of the general formula (1)

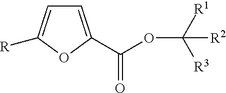

in which R=n-tetradecyl,
and R¹, R² and R³ independently are —H, a hydroxyl group, a —C₁-C₂₄ alkyl substituted by one or more hydroxyl groups
with the proviso that at most two of the groups R¹, R² and R³ represent an H atom or a hydroxyl group,
and medicinal or cosmetic topical preparations containing one or more such compounds at an effective concentration,
and the cosmetic use of one or more such compounds for reducing sebum production in human skin,
and the use of topical preparations containing one or more such compounds for the treatment of greasy and/or impure skin and/or mild forms of acne and/or greasy hair and/or greasy scalp and/or seborrhea,
remedy the disadvantages of the prior art.

The uses according to the invention follow a similar principle, since the sebaceous glands on the scalp and on the face have an identical structure and the sebum production proceeds according to the same mechanism. The application of the sebum-reducing active ingredients on the scalp region thus makes it possible to reduce sebum production and therefore prevent the cosmetically undesirable condition of greasy hair.

Preparations that are particularly advantageous according to the invention are those that are characterized in that one or more compounds of the general formula (1), advantageously in a water and/or oil phase, are present at concentrations of 0.0001-40.00% by weight, preferably 0.005-20.00% by weight, particularly preferably 0.001-6.00% by weight, in each case based on the total weight of the composition.

Preferred compounds of formula (1) are:

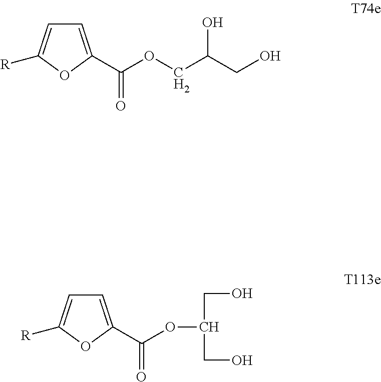

### Synthesis of TOFA (3)

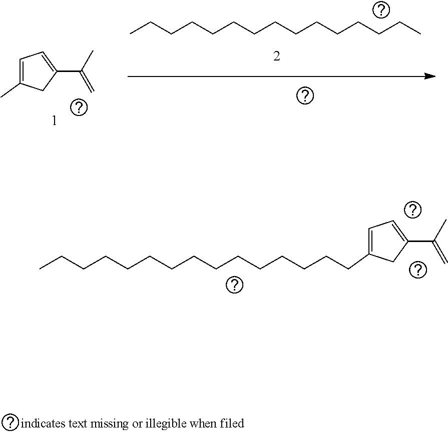

### Compound 3 (TOFA)

NaH (60% in mineral oil, 7.85 g, 196.3 mM) was added at ambient temperature to a suspension of alcohol (2) (25.3 g, 117.8 mM) in 400 ml of o-xylene and the mixture was stirred for 2 hours under reflux cooling. The mixture was cooled down to approx. 70° C., and acid (1) was added in solid form in one portion (15.0 g, 78.5 mM). The mixture was stirred for 48 h under reflux cooling and cooled to ambient temperature. The mixture was quenched with aqueous KHSO₄, extracted with EtOAc, the organic layer was additionally washed with H₂O, dried with Na₂SO₄ and evaporated. The residue was triturated with hexane, which gave Compound (3) (7.1 g, 28%). The synthesis was repeated several times in order to obtain the required amount of TOFA (approx. 100 g).

### ZE33-0116 (T113e)

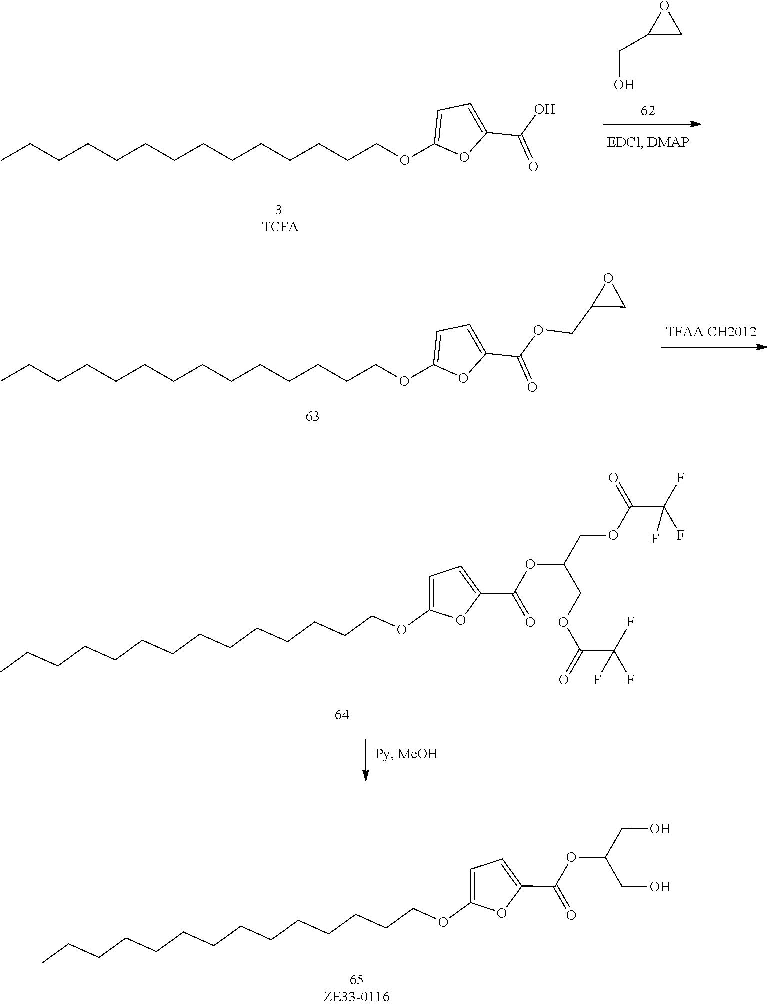 [SMILES: OCC1CO1 ; CCCCCCCCCCCCCCOc1ccc(C(=O)O)o1 ; CCCCCCCCCCCCCCOc1ccc(C(=O)OCC2CO2)o1 ; CCCCCCCCCCCCCCOc1ccc(C(=O)OC(COC(=O)C(F)(F)F)COC(=O)C(F)(F)F)o1 ; CCCCCCCCCCCCCCOc1ccc(C(=O)OC(CO)CO)o1]

### Compound (63)

Glycidol (62) (3.78 g, 51 mM) was added to a solution of TOFA (3) (13.1 g, 40 mM) in 500 ml of CH₂Cl₂, followed by the addition of EDCI (10.5 g, 55 mM) and DMAP (0.97 g, 8 mM) and the mixture was stirred at ambient temperature for 20 h. The solvent was evaporated, the residue was dissolved again in EtOAc, washed with H₂O and dried over Na₂SO₄ and evaporated. The residue was purified by column chromatography on silica gel, elution being performed with 15% EtOAc in hexane, which gave Compound (63) (9.43 g, 61%).

### Compound (64)

Trifluoroacetic anhydride (26.4 g, 126 mM) in 40 ml of CH₂Cl₂ was added at −30° C. to a solution of 9.43 g (25 mM) of Compound (63) in 120 ml of CH₂Cl₂. The mixture was stirred at ambient temperature for 4 hours and evaporated, the residue was coevaporated with CCl₄, which gave Compound (64) (14.5 g, 99%).

### Compound (65) (ZE33-0116)

A solution of pyridine (22 g) and MeOH (13 g) in 100 ml of CH₂Cl₂ was added dropwise at −55° C. to a solution of Compound (64) (14.5 g, 25 mM) in 120 ml of CH₂Cl₂. The mixture was stirred at −30° C. for 0.5 h and then at ambient temperature for 3 h. The mixture was evaporated and the residue was washed with hexane, which gave Compound (65) (ZE33-0116) (9.85 g, 100%).

### ZE33-0117 (T74e)

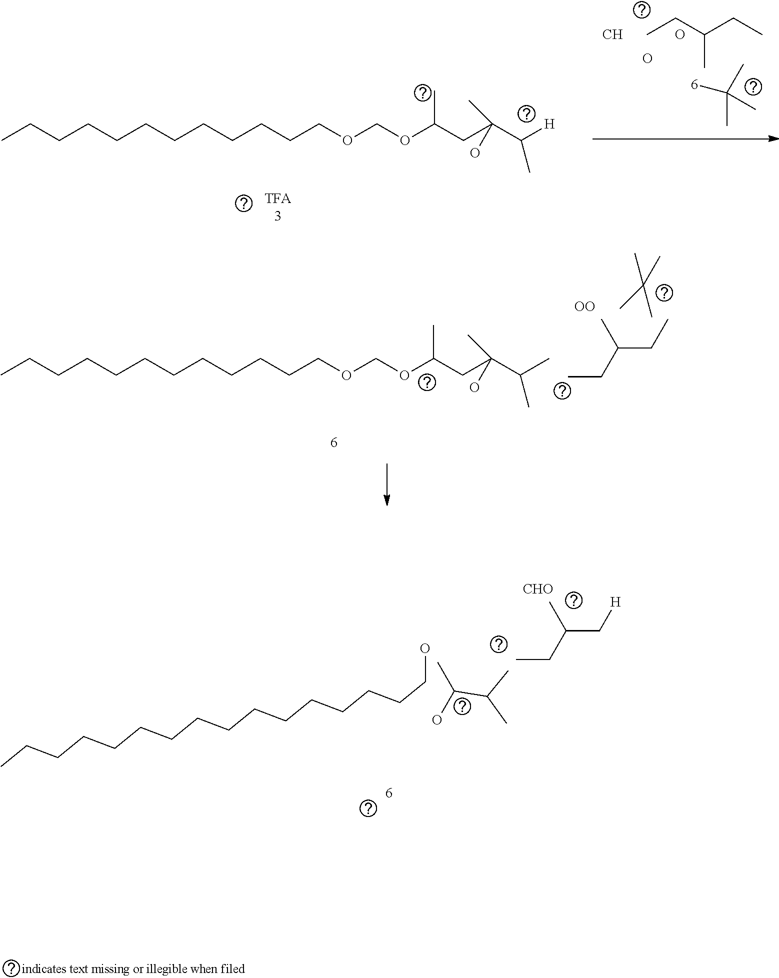

### Compound (67)

Protected glycerol (66) (4.1 g, 31 mM) was added to a solution of TOFA (3) (7.9 g, 24 mM) in 500 ml of CH₂Cl₂, followed by the addition of EDCI (6.3 g, 33 mM) and DMAP (0.61 g, 5 mM) and the mixture was stirred at ambient temperature for 20 h. The solvent was evaporated, the residue was dissolved again in EtOAc, washed with H₂O and dried over Na₂SO₄ and evaporated. The residue was purified by column chromatography on silica gel, elution being performed with 15% EtOAc in hexane, which gave Compound (67) (9.16 g, 86%).

### Compound (68) (ZE33-0117)

0.4 ml of 3 M HCl in dioxane was added to a solution of Compound (67) (8.78 g, 20 mM) in 400 ml of MeOH and the mixture was stirred at ambient temperature for 0.5 hours. The mixture was evaporated and subsequently coevaporated with MeOH to a volume of approx. 20 ml, the pellet formed was isolated by filtration and dried, which gave Compound (68) (ZE33-0117) (6.9 g, 87%).

The compounds according to the invention can advantageously be incorporated into cosmetic or dermatological preparations.

The preparations or uses according to the invention may be in the form of liquid compositions that can be applied using brushes or spreaders, roll-on devices or atomizers, as sticks and in the form of systems that can be applied from normal bottles and containers, for example creams, gels or lotions. For example, they may thus constitute a solution, an emulsion of water-in-oil (W/O) type or of oil-in-water (O/W) type, or a multiple emulsion, for example of water-in-oil-in-water (W/O/W) type, oil-in-water-in-oil (O/W/O) type, a gel, a hydrodispersion, a lamellar phase, a liquid isotropic solution phase, a micellar phase, a solid or dispersed mono- or polyhexagonal phase, a solid or dispersed mono- or polycubic phase, a lyotropic phase, a crystalline phase, a solid stick or else an aerosol.

Furthermore, the preparations or uses according to the invention may advantageously be in the form of facial toners, tinctures, cleansing formulations, pads, cotton balls or wipes, and in the form of tonics or shampoos.

Advantageously, the pH of the preparations or uses according to the invention is set in the weakly acidic to neutral range, preferably from 3.0-7.0, particularly preferably from 5.0-6.5.

The formulations according to the invention may have the customary composition and be used for the treatment of the skin and/or the hair in the sense of a dermatological treatment or a treatment in the sense of care cosmetics. They may alternatively be used in makeup products in decorative cosmetics or in the cosmetic and dermatological cleansing products.

It is of course known to those skilled in the art that cosmetic preparations are usually not conceivable without the customary auxiliaries and additives. The cosmetic and dermatological preparations according to the invention may accordingly further comprise cosmetic auxiliaries, such as those usually used in such preparations; for example consistency regulators, preservatives, stabilizers, fillers, perfumes, pigments that have a coloring effect, thickeners, suspending agents, buffer mixtures, surface-active substances, emulsifiers, softening, moistening and/or moisturizing substances, anti-inflammatory substances, additional active ingredients such as vitamins or proteins, light stabilizers, insect repellents, bactericides, water, salts, antimicrobial, proteolytic or keratolytic substances, medicaments or other customary constituents of a cosmetic or dermatological formulation such as alcohols, polyols, polymers, foam stabilizers, organic solvents or else electrolytes.

The amounts of carrier substances to be used in each case can easily be determined by those skilled in the art by simple trial and error, depending on the type of the product in question.

As customary carrier substances for the production of the preparations or uses according to the invention, it is possible to use not only water, ethanol and isopropanol, glycerol and propylene glycol, but also skincare lipids or lipoids, such as decyl oleate, cetyl alcohol, cetylstearyl alcohol and 2-octyldodecanol, in the proportions customary for such preparations, and mucilaginous substances and thickeners, for example hydroxyethylcellulose or hydroxypropylcellulose, polyacrylic acid, polyvinylpyrrolidone, but additionally also, in small amounts, cyclic silicone oils (polydimethylsiloxanes) and liquid polymethylphenylsiloxanes of low viscosity.

Emulsifiers that can be used in the preparations in a small amount, for example 1% to 6% by weight, based on the total composition, and that have proved to be suitable for the production of the preparations or uses according to the invention, which are advantageously to be applied as liquid or solid preparations to the desired skin regions, are noniogenic types, such as polyoxyethylene fatty alcohol ethers, for example cetostearyl alcohol polyethylene glycol ether with 12 or 20 added-on ethylene oxide units per molecule, cetostearyl alcohol, and sorbitan esters and sorbitan ester-ethylene oxide compounds (for example sorbitan monostearate and polyoxyethylene sorbitan monostearate), and long-chain higher molecular weight waxy polyglycol ethers. However, a whole range of other emulsifiers or emulsifier mixtures which are usually used in cosmetic preparations are additionally also suitable. These include for example, but are not restricted to, glyceryl stearate citrate, PEG-40 stearate or else polyglyceryl-3 methylglucose distearate, stearic acid, steareth-2 and steareth-21.

The oil phase of the preparations according to the invention is advantageously selected from the group of esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, from the group of esters of aromatic carboxylic acids and saturated and/or unsaturated, branched and/or unbranched alcohols having a chain length of 3 to 30 carbon atoms.

In addition, the oil phase may advantageously be selected from the group of branched and unbranched hydrocarbons and hydrocarbon waxes, silicone oils, dialkyl ethers, the group of saturated or unsaturated, branched or unbranched alcohols, and fatty acid triglycerides, namely the triglyceryl esters of saturated and/or unsaturated, branched and/or unbranched alkanecarboxylic acids having a chain length of 8 to 24, in particular 12-18, carbon atoms. The compounds of the oil phase may be of synthetic, semi-synthetic or natural origin.

Any blends of such oil and wax components may also advantageously be used in the context of the present invention.

The content of the oil phase is advantageously between 1% and 50% by weight, based on the total weight of the preparations, preferably 2%-30% by weight, especially preferably below 10% by weight.

The cosmetic and/or dermatological formulations according to the invention are used by applying a sufficient amount to the skin, scalp and/or the hair in the manner customary for cosmetics and dermatological products.

### Lipid Assay for Testing the Effectiveness of TOFA Derivatives According to the Invention

To determine the lipid-reducing effect of active ingredients, primary cell lines from sebaceous glands of different human donors are used. The cells are cultured for a total of 7 days. After the cells are seeded on day 1, the active ingredient treatment is effected on day 3 and day 5. On day 7, the cells are stained with the dye AdipoRed. This makes it possible to fluorescently stain and to quantify the lipid droplets inside the cells. The dye fluorescein diacetate (FDA), which is converted by living cells, is also used to monitor the vitality of the cells. The resulting fluorescence signal can likewise be quantified. The effect of an active ingredient can be measured and presented as the ratio of the AdipoRed and FDA fluorescence signal relative to the ratio of the corresponding solvent. The tested substances were dissolved in dimethyl sulfoxide (DMSO); all results were accordingly normalized to the DMSO-treated control.

The examples that follow are intended to elucidate, but not restrict, the invention. Unless stated otherwise, the figures are based on % by weight.

| | Example 1 | Example 2 | Example 3 | Example 4 |
|---|---|---|---|---|
| Caprylic/Capric Triglyceride | | | | |
| Propylene Glycol + Aqua | | | 5.00 | 5.00 |
| Citric Acid | | 0.00 | 0.00 | 0.00 |
| Cetearyl Alcohol | 2.00 | | | |
| Aqua + Trisodium EDTA | 1.00 | | | |
| Phenoxyethanol + Aqua | 0.40 | | | |
| Dimethicone | 3.00 | | | |
| Glycerol | 6.00 | 4.00 | 4.00 | 10.00 |
| PEG-40 Hydrogenated Castor Oil | | | 1.00 | 5.00 |
| Butylene Glycol + Aqua | | 20.00 | 10.00 | 10.00 |
| Hydroxyethylcellulose + Disodium | 0.15 | | | |
| Phosphate + Sodium Phosphate | | | | |
| Alcohol Denat. + Aqua | 8.00 | 50.00 | 40.00 | 40.00 |
| Xanthan Gum | 0.15 | | | |
| Acrylates/C10-30 Alkyl Acrylate | 0.15 | | | |
| Crosspolymer | | | | |
| PEG-150 Distearate | 0.50 | | | |
| Tapioca Starch + Aqua | 1.00 | | | |
| Ammonium Acryloyldimethyltaurate/VP | 0.50 | 0.65 | 0.65 | 0.65 |
| Copolymer + Aqua | | | | |
| Methylpropanediol | | | 4.00 | 4.00 |
| Sodium Stearoyl Glutamate + Sodium | 0.30 | | | |
| Chloride | | | | |
| TOFA derivative T74e | 2.00 | 1.00 | 2.00 | 2.00 |
| Water to | 100.00 | 100.00 | 100.00 | 100.00 |

| | Example 5 | Example 6 | Example 7 |
|---|---|---|---|
| Caprylic/Capric Triglyceride | | | 4.00 |
| Propylene Glycol + Aqua | 5.00 | | |
| Citric Acid | 0.00 | | |
| Cetyl Alcohol | | | 3.00 |
| Aqua + Trisodium EDTA | | 1.00 | 1.00 |
| Diisopropyl Adipate | | 10.00 | 10.00 |
| Phenoxyethanol + Aqua | | 0.50 | 0.80 |
| Hydrogenated Coco-Glycerides | | | 2.00 |
| Glycerol | 10.00 | 3.00 | 9.00 |
| Aqua + Sodium Hydroxide | | 0.06 | 0.01 |
| PEG-40 Hydrogenated Castor Oil | 5.00 | | |
| Butylene Glycol + Aqua | 10.00 | | 5.00 |
| Alcohol Denat. + Aqua | 40.00 | 10.00 | 5.00 |
| Xanthan Gum | | 0.30 | 0.30 |
| Acrylates/C10-30 Alkyl Acrylate | | | 0.20 |
| Crosspolymer | | | |
| Glyceryl Stearate Citrate | | | 2.00 |
| Acrylates/C10-30 Alkyl Acrylate | | 0.20 | |
| Crosspolymer | | | |
| Ammonium Acryloyldimethyltaurate/VP | 0.65 | | |
| Copolymer + Aqua | | | |
| Methylpropanediol | 4.00 | | |
| TOFA derivative T113e | 0.50 | 0.50 | 1.00 |
| Water to | 100.00 | 100.00 | 100.00 |

1.-6. (canceled)

7. A compound of general formula (1): 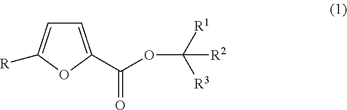
in which
R=n-tetradecyl,
and R¹, R² and R³ independently represent —H, a hydroxyl group, a —C₁-C₂₄ alkyl substituted by one or more hydroxyl groups
with the proviso that at most two of the groups R¹, R² and R³ represent an H atom or a hydroxyl group.

8. The compound of claim 7, the compound being selected from:

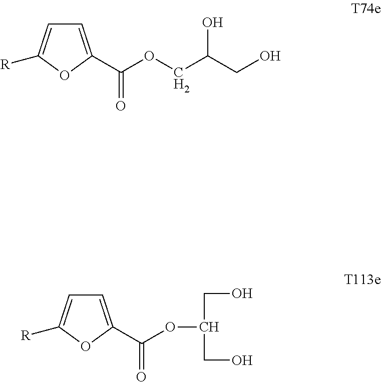

9. A medicinal or cosmetic topical preparation, wherein the preparation comprises at least one compound according to claim 7.

10. A medicinal or cosmetic topical preparation, wherein the preparation comprises at least one compound according to claim 8.

11. The preparation of claim 9, wherein the at least one compound is present in a concentration of from 0.0001% to 40.00% by weight, based on a total weight of the preparation.

12. The preparation of claim 9, wherein the at least one compound is present in a concentration of from 0.001% to 20.00% by weight, based on a total weight of the preparation.

13. The preparation of claim 9, wherein the at least one compound is present in a concentration of from 0.005% to 6.00% by weight, based on a total weight of the preparation.

14. The preparation of claim 10, wherein the at least one compound is present in a concentration of from 0.0001% to 40.00% by weight, based on a total weight of the preparation.

15. The preparation of claim 10, wherein the at least one compound is present in a concentration of from 0.001% to 20.00% by weight, based on a total weight of the preparation.

16. The preparation of claim 10, wherein the at least one compound is present in a concentration of from 0.005% to 6.00% by weight, based on a total weight of the preparation.

17. A method of reducing sebum production in human skin, wherein the method comprises applying to human skin in need thereof an effective amount of the preparation of claim 9.

18. A method of treating greasy and/or impure human skin, wherein the method comprises applying to human skin in need thereof an effective amount of the preparation of claim 9.

19. A method of treating a mild form of acne, wherein the method comprises applying to human skin in need thereof an effective amount of the preparation of claim 9.

20. A method of treating greasy hair and/or a greasy scalp, wherein the method comprises applying to hair or a scalp in need thereof an effective amount of the preparation of claim 9.

21. A method of treating seborrhea, wherein the method comprises applying to human skin in need thereof an effective amount of the preparation of claim 9.
